(19) United States
(12) Patent Application Publication
(10) Pub. No.: US 2023/0129165 A1
(43) Pub. Date: Apr. 27, 2023

(54) HYBRID COSMETIC PRODUCT CONSISTING OF CREAMY COMPACT POWDER

(71) Applicant: Art Cosmetics S.R.L., Fornovo San Giovanni (BG) (IT)

(72) Inventor: Piero Origo, Mozzanica (BG) (IT)

(73) Assignee: Art Cosmetics S.R.L., Fornovo San Giovanni (BG) (IT)

(21) Appl. No.: 17/904,895

(22) PCT Filed: Feb. 24, 2021
(86) PCT No.: PCT/IB2021/051544

§ 371 Date: Aug. 24, 2022

(30) Foreign Application Priority Data
Feb. 25, 2020 (IT) 102020000003856

(51) Int. Cl. A61K 8/02; A61K 8/25; A61K 8/29; A61K 8/44; A61K 8/36; A61K 8/362; A61K 8/58; A61K 8/892; A61K 8/34; A61K 8/37; A61Q 1/06; A61Q 1/10; A61Q 1/12

(52) U.S. Cl. CPC A61K 8/022; A61K 8/25; A61K 8/29; A61K 8/44; A61K 8/361; A61K 8/362; A61K 8/585; A61K 8/892; A61K 8/345; A61K 8/37; A61K 8/342; A61Q 1/06; A61Q 1/10; A61Q 1/12; A61K 2800/524; A61K 2800/5922; A61K 2800/872

(57) ABSTRACT

The invention relates to a cosmetic product in the form of a creamy compact powder containing a) 30-65% by weight, on the basis of the total weight of the product, of a powder phase; b) 35-70% by weight, on the basis of the total weight of the product, of a liquid binder phase consisting of one or more liquid binders. The products according to the invention have hybrid properties between compact powdered products and poured products.

### FIELD OF INVENTION

The present invention relates to a cosmetic product in the form of a creamy compact powder and the preparation process thereof.

### PRIOR ART

Known problems with compact powder products relate to the tendency of the powder to scatter out of the container, a phenomenon also called “fallout”, during the stage of taking up/application with an applicator, such as a make-up brush, causing the powder to fall onto the user's face and clothes, and the tendency of the product surface to “glaze” if a large amount of liquid binder phase is included.

Conversely, anhydrous poured products containing large percentages of waxy lipid binder phase can exhibit defects like transudation, ie. formation of microdrops on the surface, which not only make the product unattractive, but also reduce its application performance and residence time on the skin because the product is applied unevenly. Anhydrous poured products can also exhibit the phenomenon of crystallisation of some raw materials commonly used in their formulation, for example waxes such as microcrystalline, carnauba or candelilla wax. Crystal formation makes the surface of the product rough and coarse, and therefore unacceptable to consumers.

Other defects which may be perceived in anhydrous poured products may derive from pouring points, such as uneven surface colour taking the form of streaks or small circles of darker or lighter colour on the surface.

The product according to the present invention does not suffer from such defects and, due to its cream consistency, does not exhibit the fallout phenomenon described above, which is typical of compact powders. Equally, it does not exhibit glazing, although the percentages of liquid binder phase in the formulation are much higher than in conventional compact powders in a tray: up to 20-25% more relative to the total weight of the product.

### DESCRIPTION OF THE INVENTION

The subject of the present invention is a cosmetic product in the form of a creamy compact powder containing:

a) 30-65% by weight, on the basis of the total weight of the product, of a powder phase;

b) 35-70% by weight, on the basis of the total weight of the product, of a liquid binder phase consisting of one or more liquid binders.

The product according to the invention preferably does not include a solid fatty phase, and does not include water.

The powder phase is preferably present in a percentage ranging between 45% and 60% by weight of the total weight of the product.

The ratio between powder phase and liquid binder phase preferably ranges between 1.8:1 and 1:1.

“Powder phase” means a phase consisting of solid compounds and mixtures thereof, the melting point of which is greater than 250° C.

Said powder phase consists of mica, fluorphlogopite, silica, metal stearates, boron nitride, silicone powders such as elastomers, resins such as trimethylsiloxysilicate, polymethylsilsesquioxane and hybrid powders, cellulose and derivatives thereof, polyurethanes, natural and synthetic waxes in micronised form, sodium aluminium sulphosilicate complexes, zeolite, hectorite, lauroyl lysine, boron nitride, and mixtures thereof, pigments such as iron oxides, ferric ferrocyanide, ultramarine blue, ultramarine pink, manganese violet, titanium dioxide, cochineal, lakes, pearl dyes and synthetic azo dyes such as yellow 5 lake-IC 19140, triphenylmethane dyes such as blue 1 lake-IC 42090, and xanthenes such as yellow 8-CI45350.

More preferably, the powder phase consists of mica, fluorphlogopite, silicone powders, silica, metal stearates, zeolite, hectorite, lauroyl lysine, boron nitride, trimethylsiloxysilicate or mixtures thereof.

The cosmetic product according to the invention preferably contains a liquid binder phase in a percentage ranging between 40% and 55% by weight of the total weight of the product.

The liquid binder phase consists of one or more emollient liquids and wetting agents usable in the cosmetic field.

Examples of said emollient liquids and wetting agents usable in the liquid binder phase are non-volatile silicones, such as straight-chain polydimethylsiloxane with a viscosity ranging from 5 cSt to 1000000 cSt, silicone rubbers such as dimethiconol, phenylsiloxanes, elastomers in gel form, glycerin, paraffin hydrocarbons obtained from petroleum, such as liquid paraffin, vaseline or paraffin waxes; glycerol esters such as capric/caprylic triglyceride, triethylhexanoin, triolein and triisostearin; non-glyceryl esters such as cetyl lactate, ethylhexyl palmitate and cetearyl ethylhexanoate; glyceryl ethers such as ethylhexylglycerin; lanolin and derivatives thereof, such as hydroxylated lanolin; natural oils, such as macadamia oil, sweet almond oil, sunflower oil, etc.

The liquid binder phase can also include, possibly in smaller percentages, polyols with wetting activity such as butylene glycol, propanediol, methyl propanediol and 1,2-hexanediol or pentylene glycol, sorbitol or glycerin.

The preferred emollients and wetting agents usable in the liquid binder phase are dimethicone, dimethiconol, capric/caprylic triglyceride, octyldodecyl stearoyl stearate, ethylhexyl palmitate, hydrogenated polydecene, squalane, triethylhexanoin, pentaerythrityl tetraisostearate, octyldodecanol, isononyl isononanoate, cetearyl ethylhexanoate, 1,2-hexanediol and ethylhexylglycerin.

The cosmetic product according to the invention can also contain preservatives such as organic acids like dehydroacetic acid or sorbic acid; organic salts such as potassium sorbate or sodium dehydroacetate; sunscreens, extracts and active ingredients.

A preferred formulation of the cosmetic product in creamy compact powder form according to the invention contains:

| | % (w/w) |
|---|---|
| POWDER PHASE (PHASE A) | |
| MICA | 0.1-50% |
| IRON OXIDES (CI 77499) | 0.1-40% |
| FERRIC FERROCYANIDE (CI77510) | 0.1-40% |
| TITANIUM DIOXIDE (CI 77891) | 0.1-40% |
| SILICA | 0.1-20% |
| SYNTHETIC FLUORPHLOGOPITE | 0.1-50% |
| LAUROYL LYSINE | 0.1-15 |
| ZINC STEARATE | 0.1-10% |
| DEHYDROACETIC ACID (preservative) | 0.1-0.5% |
| TRIMETHYLSILOXYSILICATE | 0.1-10% |
| VINYL DIMETHICONE/METHICONE | 0-3% |
| SILSESQUIOXANE CROSSPOLYMER | |
| BINDER PHASE (PHASE B) | |
| GLYCERINE | 0-10% |
| PENTYLENE GLYCOL | 0-3% |
| DIMETHICONE & DIMETHICONOL | 0.1-30% |
| ETHYLHEXYLGLYCERIN | 0.1-2% |
| ETHYLHEXYL PALMITATE | 0.1-15% |
| CAPRYLIC/CAPRIC TRIGLYCERIDE | 0.1-15% |
| OCTYLDODECANOL | 0.1-15% |

The cosmetic products according to the invention can be used as make-up products such as foundation, lipstick, eyeliner, eyeshadow, face powder, earth, eyebrow pencil, highlighter, blusher and corrector, which can be characterised by different finishes, such as matte, demi-matte, satin-finish, pearly, shimmer, metallic and glitter.

The products according to the invention have hybrid properties between compact powdered products and poured products. Poured products are defined as mixtures of generally lipid substances which are liquid at high temperature and solidify at room temperature after pouring into containers such as sticks, trays or jars.

In fact, the cosmetic products according to the present invention have considerably superior properties to the known compact powdered cosmetic products due to their high creaminess, colour release ability and skin adherence. Said properties give rise to greater colour saturation of the applied product and a greater film thickness of the applied product. The cosmetic products according to the invention, when applied to the skin, conceal skin blemishes such as rosacea or discoloured skin, maintaining considerable adherence over time.

Moreover, the products according to the invention do not exhibit the problem of fallout or glazing of compacted product.

The reduction in fallout is known to be directly proportional to the increase in the liquid binder phase, but it is also known that in conventional compact powders, glazing increases in direct proportion to the increase in the liquid binder phase. The product according to the invention does not present fallout or glazing problems despite the fact that it incorporates a much larger percentage of liquid binder phase than conventional compact powders. This is due to the particular manufacturing method which gives rise to total saturation of the powder grain with the liquid binder phase, and therefore produces a product that is creamier and more homogeneous throughout its use.

The product according to the invention is obtained by a process comprising the following steps:

i) mixing the powder phase with the liquid binder phase under vacuum at 0.3-1 bars, preferably between 0.3 and 0.5 bars, for a time ranging from 1 to 72 hours, preferably 1 to 3 hours, at a temperature ranging between 20° C. and 60° C., preferably between 20° C. and 30° C.;

ii) saturating and stabilising the mixture for 2-72 hours under vacuum at 0.3-1 bars and at a controlled temperature of 20-60° C., preferably at 0.3-0.5 bars and a temperature of 30-50° C.;

iii) adding an amount of volatile solvent equal to 10-40% of the total weight of the product, and filling containers or trays with the mixture;

iv) compressing the mixture at a controlled pressure of 2-200 bars;

v) heating the product for 2-72 hours at a controlled temperature ranging between 30 and 50° C. to extract the volatile solvent part of the mixture.

The residual volatile solvent must not exceed 2-3% of the weight of the finished product after baking.

The presence of an amount of volatile solvent ranging between 10 and 40% by weight of the paste before baking gives rise to the viscosity necessary to terminate the mixing step in a mixer such as a vacuum planetary mixer which, due to its dual rotatory movement, optimally homogenises the product.

Steps i) and ii) prior to the addition of the volatile solvent take place at a controlled temperature of 20-60° C., under a vacuum of 0.3-1 bars (preferably 0.3-0.5 bars), for a time ranging from 2 to 72 hours. This is crucial to the process of saturation of the powder phase with the liquid binder phase which, without interference by air and volatile solvent, completely wets the powder grain which, by the end of the process, has no more absorption capacity. The phase of saturation of the powder with the liquid binder phase takes place under controlled pressure and temperature conditions in order to accelerate the process. It is essential for the volatile solvent to be added at a second stage to prevent large amounts thereof from being absorbed by the powder grains, as they are already saturated with liquid binder phase. Said addition of volatile solvent must take place rapidly because said volatile solvent would tend to “scour” the powder from the liquid binder phase over time, taking its place and thereby exposing it to greater mechanical absorption by the screens during the stage of compacting in trays. “Rapidly” here means compacting the product within eight hours of tray-filling, and preferably within 5 hours.

“Volatile solvent” here means one or more substances which present high vapour pressure at ambient pressure and temperature. https://it.wikipedia.org/wiki/Pressionehttps://it.wikipedia.org/wiki/Temper aturahttps://it.wikipedia.org/wiki/Tensione_di_vapore

The volatile solvent can be selected from hydrocarbons, straight-chain and cyclic silicones or water.

The method described above causes the majority of the liquid binder phase to be absorbed into the powder, giving rise to a final softer, creamier, more adhesive texture with better colour release than conventional compacted powders.

In step iii), when the desired consistency has been obtained, containers called “trays”, made of various materials (metal, earthenware, plastic or silicone, such as tinplate, aluminium, polycarbonate, polyurethane, acrylic derivatives, etc.), are filled with the mixture.

The choice of material used to make the container or tray depends on the type of solvent used, and the intended shape of the product.

In step iv), the product poured into the tray undergoes compression at 2-200 bars, giving rise to a product with a structure and consistency that allow safe handling during the stages of sale and transport to destination and use by the consumer.

During this step, one or more tissues, called “canvas”, with absorption ranging between 20 and 500 g/m², are laid on the product. The purpose of the tissues is to promote the process of compression and absorption of the product and detachment thereof from the mould. They can be made of various materials, such as polyamides, polyurethanes, trinitrotoluene, paper, and the like.

The product poured into the tray preferably undergoes compression of 5-110 bars with specific tissues having an absorption ranging between 35 and 350 g/m².

Alternatively, the mixture can be poured into moulds, in order to pre-form the shape of the finished product; said shape will then be deposited on a tray, and then undergo compression and heating as described above.

In step v), the compacted product is then heated to a temperature ranging between 30 and 50° C. The heating temperature depends on the solvent used. The heating can be performed by one of the conventional methods such as a microwave oven, controlled humidity oven or other type. Baking must continue for long enough to reach a residual solvent level <3% by weight of the total weight of the finished product. In general, the heating time ranges between 2 and 72 hours, depending on the solvent used and its initial amount, preferably between 12 and 48 hours.

The resulting product is cleaned (ie. any surplus product on the surface or edges of the container in which the cosmetic is housed is removed), and packaged in the final sales packaging.

The final product is characterised by a consistency ranging between a compact powder and an anhydrous poured product, as demonstrated by the TGA analyses conducted on the products according to the invention and shown in the examples below.

The resulting product has a highly creamy consistency which inhibits the fallout phenomenon described above; moreover, unlike the classic compact powder products, the feel of this novel product resembles that of a soft, uniform, elastic “paste” to the touch, but it does not suffer from glazing. The product glides easily to the touch, giving a smooth, velvety sensation.

Unlike the classic compact powders, which require repeated applications to give a good pigmenting effect, in order to intensify the colour result and film thickness, the products according to the invention chromatically mask skin blemishes such as rosacea and discoloured skin by providing greater colour saturation with a single application. This property leads to a marked saving of time, with a more lasting result.

Unlike the classic compact powders which, as already stated, must be applied several times to obtain the optimum application/colour result, thus rendering the film thick and rather unattractive, the products according to the invention, due to their elastic consistency, give the same effect, if not better, while maintaining a very slight thickness of the product on the skin, leading to greater breathability of the skin and a lower impression of “heaviness” during the day.

The classic compact powders in trays contain, by nature, a very small percentage of liquid binder phase, not exceeding 15-25% by weight; although this special feature makes them suitable for application to combination or greasy skin, it drastically reduces the persistence of the product once applied; “persistence” here refers to the residence time of the product and above all the duration of its colour intensity, from the time when it is applied to the face/body.

The novel product, characterised by an elastic matrix consisting of a mixed powder phase/liquid phase, gives rise to a uniform structure, wherein the powders are perfectly saturated and “enveloped” in the liquid fraction.

This condition has three advantages:
a) reduction of air spaces between the powder particles, which gives rise to a more homogeneous/uniform product;
b) greater ability of the powder particles to adhere to the skin, as they are thoroughly “enveloped” in and saturated by the liquid binder phase;
c) greater ability to add liquid binder phase to the product, thereby enhancing its emollience.

### Example 1—Foundation

A hybrid foundation (according to the invention), a compact foundation (comparator) and a poured foundation (comparator) having the following formulations were prepared (the percentages are given by weight):

%

COMPACT FOUNDATION

| | |
|---|---|
| PHASE A | |
| TALC | 0.8/100 |
| BORON NITRIDE | 0.5-4 |
| LAUROYL LYSINE | 0.5-4 |
| HDI/TRIMETHYLOL HEXYLLACTONE CROSSPOLYMER | 0.5-5 |
| ZINC STEARATE | 0.5-4 |
| PHASE B | |
| IRON OXIDES (CI 77491) | 0.1-5 |
| IRON OXIDES (CI 77492) | 0.1-5 |
| IRON OXIDES (CI 77499) | 0.1-5 |
| RED 7 LAKE (CI 15850) | 0.1-5 |
| PHASE C | |
| ISODECYL NEOPENTANDATE | 0.5-5 |
| OCTYLDODECYL STEAROYL STEARATE | 0.5-5 |
| DIMETHICONE | 0.5-5 |

HYBRID FOUNDATION

| | |
|---|---|
| PHASE A | |
| MICA | 0.8/100 |
| IRON OXIDES (CI 77499) | 5-10 |
| FERRIC FERROCYANIDE (CI77510) | 5-10 |
| TITANIUM DIOXIDE (CI 77789) | 15-20 |
| SILICA | 3-7 |
| SYNTHETIC FLUORPHLOGOPITE | 1-10 |
| DEHYDROACETIC ACID | 0.25 |
| PHASE B | |
| DIMETHICONE | 5-10 |
| CAPRYLIC/CAPRIC TRIGLYCERIDE | 5-20 |
| HECTORITE | 5-15 |
| DIMETHICONOL | 0.5-5 |
| DIMETHICONE/VINYL DEMETHICONE CROSSPOLYMER | 5-20 |

POURED FOUNDATION

| | |
|---|---|
| PHASE A | |
| DICAPRYLYL CARBONATE | 20-40 |
| PHENYL TRIMETHICONE | 10-25 |
| HYDROGENATED POLYDECENE | 10-20 |
| Bis-Diglyceryl Polyacyladipate-2 | 10-20 |
| PHASE B | |
| POLYETHYLENE | 2-10 |
| BEESWAX | 2-10 |
| PHASE C | |
| IRON OXIDES | 0.1-5 |
| IRON OXIDES | 0.1-5 |
| IRON OXIDES | 0.1-5 |
| SILICA | 0.1-5 |
| PHASE D | |
| HDI/TRIMETHYLOL HEXYLLACTONE CROSSPOLYMER | 0.1-5 |
| POLYMETHYL METHACRYLATE | 0.1-5 |

In the comparator compact foundation, the powder phase is present in an amount ranging between 75 and 98%, and the liquid phase is present in an amount ranging between 2 and 15%

In the comparator poured foundation, the powder phase is present in an amount ranging between 0.1 and 10%, and the liquid phase is present in an amount ranging between 90 and 99.9%

In the hybrid foundation according to the invention, the powder phase is present in an amount ranging between 30 and 65%, and the liquid phase is present in an amount ranging between 35 and 70%.

Preparation Method of Compact Foundation (Comparator)

Phase A was loaded into a mill and mixed for 5-10 minutes at 3400/3600 rpm.

Phase B was then loaded into the mill and mixed for 5-10 minutes at 3400/3600 rpm.

Phase C was then loaded into the mill and mixed for 5-10 minutes at 3400/3600 rpm.

The resulting powder was sieved with an automatic vibrosieve, using a metal screen with 90/180 mesh.

The powder was compacted in suitable metal containers (trays), generally made of tinplate and/or aluminium.

Preparation Method of Hybrid Foundation (According to the Invention):

Phase A+B was loaded into a mixer and mixed for 12-48 hours at 200/800 rpm, 0.3-2 bars, for 1-3 hours.

The powder was left to saturate in the mixer for 12-48 hours under vacuum at 0.3-2 bars, and at a controlled temperature of 20-60° C.

10-40% of solvent was then poured into the mixer in a thin stream and the mixer was run for 5-15 minutes at 200/800 rpm, at a controlled temperature of 20-30° C.

The resulting powder was compacted in suitable metal containers (trays), generally made of tinplate and/or aluminium.

The resulting compacted powder was placed in an oven heated to a temperature ranging between 30 and 50° C. for a time ranging between 2 and 72 hours.

Preparation Method of Poured Foundation (Comparator):

Phase A was loaded into a melter, and the temperature was kept constant at between 65 and 95° C.

Phase B was added to the melter, and the temperature was kept constant at between 65 and 95° C.

Phase C was added to the melter, and the temperature was kept constant at between 65 and 95° C.

Phase D was then added to the melter, mixed and poured into trays.

### Example 2—Eyeshadow

A hybrid eyeshadow (according to the invention), a compact eyeshadow (comparator) and a poured eyeshadow (comparator) having the following formulations were prepared (the percentages are given by weight):

%

COMPACT EYESHADOW

| | |
|---|---|
| PHASE A | |
| TALC | 0.8/100 |
| NYLON 12 | 0.5-5 |
| DEHYDROACETIC ACID | 0.4 |
| HDI/TRIMETHYLOL HEXYLLACTONE CROSSPOLYMER | 0.5-5 |
| ZINC STEARATE | 0.5-4 |
| SILICA | 0.5-8 |
| PHASE B | |
| IRON OXIDES (CI 77491) | 5-10 |
| IRON OXIDES (CI 77492) | 5-10 |
| IRON OXIDES (CI 77499) | 5-10 |
| PHASE C | |
| CAPRYLIC/CAPRIC TRIGLYCERIDE | 0.5-8 |
| DIMETHICONE | 0.5-8 |

HYBRID EYESHADOW

| | |
|---|---|
| PHASE A | |
| MICA | 0.8/100 |
| IRON OXIDES (CI 77499) | 5-10 |
| FERRIC FERROCYANIDE (CI77510) | 5-10 |
| TITANIUM DIOXIDE (CI 77789) | 15-20 |
| SILICA | 3-10 |
| ALLUMINUM POWDER | 5-20 |
| DEHYDROACETIC ACID | 0.25 |
| PHASE B | |
| DIMETHICONE | 5-20 |
| CAPRYLIC/CAPRIC TRIGLYCERIDE | 5-20 |
| TRIMETHYLSILOXYSILICATE | 1-8 |
| DIMETHICONOL | 0.5-5 |

POURED EYESHADOW

| | |
|---|---|
| PHASE A | |
| POLYETHYLENE | 20-40 |
| BEESWAX | 10-25 |
| VP/Eicosene Copolymer | 0.1-5 |
| Disteardimonium Hectorite | 0.1-5 |
| ISODODECANO | 10-25 |
| TRIMETHYLSILOXYSILICATE | 0.1-8 |
| PHASE B | |
| IRON OXIDES | 0.1-5 |
| IRON OXIDES | 0.1-5 |
| IRON OXIDES | 0.1-5 |

In the comparator compact eyeshadow, the powder phase is present in an amount ranging between 75 and 98%, and the liquid phase is present in an amount ranging between 2 and 15%.

In the comparator poured eyeshadow, the powder phase is present in an amount ranging between 0.1 and 10%, and the liquid phase is present in an amount ranging between 90 and 99.9%.

In the hybrid eyeshadow according to the invention, the powder phase is present in an amount ranging between 30 and 65%, and the liquid phase is present in an amount ranging between 35 and 70%.

Preparation Method of Compact Eyeshadow (Comparator):

Phase A was loaded into a mill and mixed for 2-10 minutes at 3400/3600 rpm.

Phase B was then loaded into the mill and mixed for 2-10 minutes at 3400/3600 rpm.

Phase C was then loaded into the mill and mixed for 2-10 minutes at 3400/3600 rpm.

The resulting powder was sieved with an automatic vibrosieve, using a metal screen with 20/120 mesh.

The powder was compacted in suitable metal containers (trays), generally made of tinplate and/or aluminium.

Preparation Method of Hybrid Eyeshadow (According to the Invention):

Phase A+B was loaded into a mixer and mixed for 12-48 hours at 200/800 rpm, 0.3-2 bars, for 1-3 hours.

The powder was left to saturate in the mixer for 12-48 hours under vacuum at 0.3-2 bars, and at a controlled temperature of 20-60° C.

10-40% of solvent was then poured into the mixer in a thin stream and the mixer was run for 5-15 minutes at 200/800 rpm, at a controlled temperature of 20-30° C.

The resulting powder was compacted in suitable metal containers (trays), generally made of tinplate and/or aluminium.

The resulting compacted powder was placed in an oven heated to a temperature ranging between 30 and 50° C. for a time ranging between 2 and 72 hours.

Preparation Method of Poured Eyeshadow (Comparator):

Phase A was loaded into a turboemulsifier, and the temperature was kept constant at between 65 and 95° C.

Phase B was added to the turboemulsifier, and the temperature was kept constant at between and 95° C.

The molten mixture was then poured into trays.

### Example 3—Thermogravimetric Analysis of the Products of Example 1

Thermogravimetric analysis was used to analytically characterise the products obtained in Examples 1 (foundation) and 2 (eyeshadows), ie. compact, hybrid and anhydrous poured powders.

The technique involves continuous measurement over time of the variation in mass of a sample of material as a function of time (isotherm) or temperature (heating/cooling ramp), under controlled inert, reducing or oxidising atmospheric conditions. The result of said analysis, generally denoted as the Thermogram or Thermal Degradation Curve, is a graph showing the weight variation (axis Y), as an absolute value or percentage, as a function of time or temperature (axis X).

Three foundations obtained as reported in Example 1 underwent thermogravimetric analysis: one compact (code 65084C01), one hybrid (code 19P63MV10M2-BK) and one poured (code 137181).

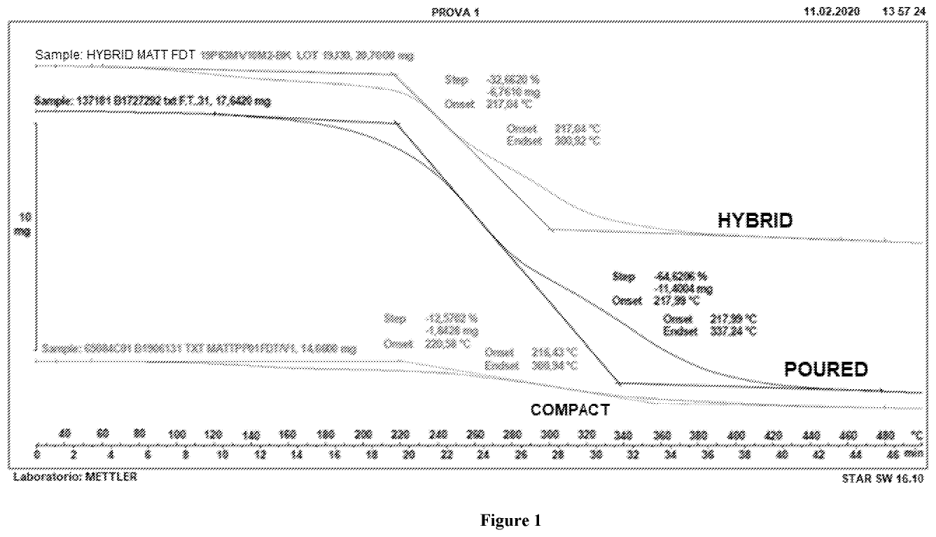

The results of the thermogravimetric analysis on the products of Example 1 are set out in FIG. 1.

The thermograms of the poured foundation and the hybrid foundation exhibit similar trends, whereas the compact foundation exhibits a more linear trend over time.

If the weight loss percentages are calculated, it will be seen that at temperatures ranging between 60 and 380° C., the compact foundation (code 65084C01) exhibits a weight loss of −12.5702%, the hybrid foundation (code 19P63MV10M2-BK) exhibits a weight loss of −32.6620%, and the poured foundation (code 137181) exhibits a weight loss of −64.6206%.

### Example 4—Thermogravimetric Analysis of the Products (Eyeshadows) of Example 2

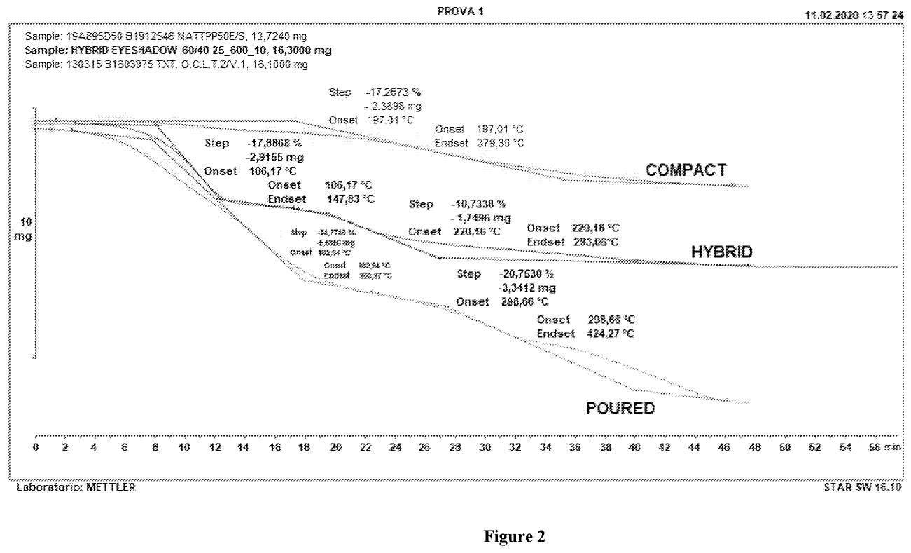

The thermogravimetric analysis of the three eyeshadows obtained in Example 2 is shown in FIG. 2.

The thermogravimetric analyses confirm the same trend as for the foundations, as the thermograms of the hybrid and poured eyeshadows are similar. When the percentage weight losses are calculated, the compact product exhibits a weight loss of −17.2673%, the hybrid product exhibits a weight loss of −28.6206% (value obtained from the sum of the two relative peaks of −17.8868% and −10.7338%), and the poured product exhibits a weight loss of −55.527% (value obtained from the sum of the two relative volatility peaks of −34.7740% and −20.7530%).

These figures analytically demonstrate that the hybrid powder absorbs a liquid binder phase in a higher amount than a traditional compact powder, exhibiting a texture with behaviour positioned between that of a compact powder and an anhydrous poured powder, but with a performance superior to both products.

1. A cosmetic product in the form of a creamy compact powder containing:
a) 30-65% by weight, on the basis of the total weight of the product, of a powder phase;
b) 35-70% by weight, on the basis of the total weight of the product, of a liquid binder phase consisting of one or more liquid binders.

2. The cosmetic product according to claim 1 wherein the powder phase ranges between 45% and 60% by weight of the total weight of the product.

3. The cosmetic product according to claim 1 wherein the powder phase consists of mica, fluorphlogopite, silica, metal stearates, boron nitride, silicone powders such as elastomers, resins such as trimethylsiloxysilicate, polymethylsilsesquioxane and hybrid powders, sodium aluminium sulphosilicate complexes, zeolite, hectorite, lauroyl lysine, boron nitride and mixtures thereof, pigments such as iron oxides, ferric ferrocyanide, ultramarine blue, ultramarine pink, manganese violet, titanium dioxide, cochineal, lakes, pearl dyes and synthetic azo dyes such as yellow 5 lake-IC 19140, triphenylmethane dyes such as blue 1 lake-IC 42090, and xanthenes such as yellow 8-CI45350.

4. The cosmetic product according to claim 3 wherein the powder phase consists of mica, fluorphlogopite, silicone powders, silica, metal stearates, zeolite, hectorite, lauroyl lysine, boron nitride, trimethylsiloxysilicate or mixtures thereof.

5. The cosmetic product according to claim 1 wherein the liquid binder phase ranges between 40% and 55% by weight of the total weight of the product.

6. The cosmetic product according to claim 1 wherein the liquid binder phase consists of one or more usable emollient liquids and wetting agents selected from non-volatile silicones such as straight-chain polydimethylsiloxane with viscosity ranging from 5 cSt to 1000000 cSt, silicone rubbers such as dimethiconol, phenylsiloxanes, elastomers in gel form, glycerin, paraffin hydrocarbons obtained from petroleum, such as liquid paraffin, vaseline or paraffin waxes; glyceryl esters such as capric/caprylic triglyceride, triethylhexanoin, triolein and triisostearin; non-glyceryl esters such as cetyl lactate, ethylhexyl palmitate and cetearyl ethylhexanoate; glyceryl ethers such as ethylhexylglycerin; lanolin and derivatives thereof, such as hydroxylated lanolin; natural oils, such as macadamia oil, sweet almond oil or sunflower oil.

7. The cosmetic product according to claim 6 wherein the emollients and wetting agents usable in the liquid binder phase are dimethicone, dimethiconol, capric/caprylic triglyceride, octyldodecyl stearoyl stearate, ethylhexyl palmitate, hydrogenated polydecene, squalane, triethylhexanoin, pentaerythrityl tetraisostearate, octyldodecanol, isononyl isononanoate, cetearyl ethylhexanoate, 1,2-hexanediol or ethylhexylglycerin.

8. The cosmetic product according to claim 6 wherein the liquid binder phase also comprises percentages of polyols with wetting activity.

9. The cosmetic product according to claim 1 also containing preservatives such as organic acids like dehydroacetic acid or sorbic acid; organic salts such as potassium sorbate or sodium dehydroacetate; sunscreens, extracts and active ingredients.

10. The cosmetic product according to claim 1 containing:

| | % (w/w) |
|---|---|
| POWDER PHASE (PHASE A) | |
| MICA | 0.1-50% |
| IRON OXIDES (CI 77499) | 0.1-40% |
| FERRIC FERROCYANIDE (CI77510) | 0.1-40% |
| TITANIUM DIOXIDE (CI 77891) | 0.1-40% |
| SILICA | 0.1-20% |
| SYNTHETIC FLUORPHLOGOPITE | 0.1-50% |
| LAUROYL LYSINE | 0.1-15 |
| ZINC STEARATE | 0.1-10% |
| DEHYDRO ACETIC ACID (preservative) | 0.1-0.5% |
| TRIMETHYLSILOXYSILICATE | 0.1-10% |
| VINYL DIMETHICONE/METHICONE | 0-3% |
| SILSESQUIOXANE CROSSPOLYMER | |
| BINDER PHASE (PHASE B) | |
| GLYCERINE | 0-10% |
| PENTYLENE GLYCOL | 0-3% |
| DIMETHICONE & DIMETHICONOL | 0.1-30% |
| ETHYLHEXYLGLYCERIN | 0.1-2% |
| ETHYLHEXYL PALMITATE | 0.1-15% |
| CAPRYLIC/CAPRIC TRIGLYCERIDE | 0.1-15% |
| OCTYLDODECANOL | 0.1-15% |

11. The cosmetic product according to claim 1 in the form of foundation, lipstick, eyeliner, eyeshadow, face powder, earth, eyebrow pencil, highlighter, blusher and corrector, characterised by different finishes, such as matte, demi-matte, satin-finish, pearly, shimmer, metallic and glitter.

12. A process for the preparation of the cosmetic product according to claim 1, comprising the steps of:
i) mixing the powder phase with the liquid binder phase under vacuum at 0.3-1 bars for a time ranging from 1 to 72 hours at a temperature ranging between 20° C. and 60° C., preferably between 20° C. and 30° C.;
ii) saturating and stabilising the mixture for 2-72 hours under vacuum at 0.3-1 bars and at a controlled temperature of 20-60° C.;
iii) adding an amount of volatile solvent equal to 10-40% of the total weight of the product and filling containers or trays with the mixture;
iv) compressing the mixture at a controlled pressure of 2-200 bars;
v) heating the product for 2-72 hours at a controlled temperature of 30-50° C. to obtain an amount of residual volatile solvent not exceeding 2-3% of the weight of the final product.

13. The process according to claim 12 wherein the volatile solvent is selected from hydrocarbons, straight-chain and cyclic silicones, and water.

14. A cosmetic product in the form of a creamy compact powder containing:
a) 30-65% by weight, on the basis of the total weight of the product, of a powder phase;
b) 35-70% by weight, on the basis of the total weight of the product, of a liquid binder phase consisting of one or more liquid binders; obtained by the process according to claim 13.

15. The process according to claim 12, wherein in step i) the powder phase is mixed with the liquid binder under vacuum between 0.3 and 0.5 bars.

16. The process according to claim 12, wherein in step i) the powder phase is mixed with the liquid binder for a time ranging between 1 to 3 hours.

17. The process according to claim 12, wherein in step i) the powder phase is mixed with the liquid binder at a temperature ranging between 20° C. and 30° C.

18. The process according to claim 12, wherein in step ii) the mixture is saturated and stabilized under vacuum at 0.3-0.5 bars and at a temperature of 30-50° C.
